(19) United States
(12) Patent Application Publication
(10) Pub. No.: US 2010/0041965 A1
(43) Pub. Date: Feb. 18, 2010

(54) Handheld Sleep Assistant Device and Method

(75) Inventors: Shih-Chung Kang, Taipei (TW); Rayleigh Ping-Ying Chiang, Taipei City (TW); Peter Liu, Taipei (TW); Zai-Ting Yeh, Taipei (TW); Chia-Hsuan Chiang, Taipei (TW); Hung Lin Chi, Taipei (TW); Yu Ting Yeh, Taipei (TW); I-Ling Chen, Taipei (TW)

Correspondence Address: BEVER HOFFMAN & HARMS, LLP;901 Campisi Way, Suite 370, Campbell, CA, 95008, US

(73) Assignee: National Taiwan University, Taipei (TW)

(21) Appl. No.: 12/537,971

(22) Filed: Aug. 7, 2009

(30) Foreign Application Priority Data
Aug. 14, 2008 (TW) 097131038

(51) Int. Cl. A61B 5/00

(52) U.S. Cl. 600/301

(57) ABSTRACT

In accordance with one aspect of the present invention, an electrical sleep assistant device, which comprises a handheld case, a display panel, a plurality of biosensors, and a calculation module, is provided. The case has a surface. The display panel is disposed on the surface of the case. The plurality of biosensors are disposed on the surface of the case for collecting a plurality of physiological information. The calculation module is disposed in the case, and coupled to the display panel and the plurality of biosensors. Preferably, a plurality of buttons disposed on the surface of the case, and coupled to the calculation module.

### FIELD OF THE INVENTION

The present invention relates to a sleep assistant device and the assistant method thereof, and more particularly to a handheld electrical sleep assistant device and the assistant method thereof.

### BACKGROUND OF THE INVENTION

Through out the entire life span of a human being, one third of the time is for sleeping. Therefore, sleeping is very important. A quality life is usually based upon high quality of sleeping. Thus, the quality of sleeping is fundamental for sustaining the quality of life. However, according to a research result, 11.76 percent (about 32 million) of the American people suffer insomnia. Population of those patients are distributed in different ages, sexes, races and social hierarchies. Accordingly, it is necessary for the research to resolve sleep problems and produce the environment for high quality sleeping.

In general, it is easier for people to fall asleep under a comfortable, safety and familiar situation. One of the methods for resolving sleeping issue is to construct the so-called “best sleep environment”. Nonetheless, the development for constructing the best sleep environment is still focused on the developing and integration of monitoring tools of biomedical engineering, which take advantage of biosensors for monitoring a variety of indices and recording data such as brainwave, breathing, snoring, muscle tension and blood oxygenation to facilitate the doctors in examining different kinds of sleeping obstacles and verifying the improvement conditions after a treatment.

Mainly designed for clinical usages, the present sleep assistant devices are configured to receive the physiological information of a user, analyze the physiological information with a personal computer, and implement consequent sleep assistant methods by means of computers. However, the abovementioned sleep assistant device inevitably has many deficiencies in terms of application, for example: (1) The whole apparatus is so bulky that it can not be portable; (2) With the use of too many biosensors of profession class, the mentioned sleep assistant devices can be operated by the skilled people in this art only. Without the assistance from the skilled people, the ordinary people will have a hard time to be acquainted with those biosensors of profession class; (3) Due to too many biosensors of profession class, the difficulty of assembly/disassembly is increases, the convenience for use is highly reduced, and even result in obstacles for use; (4) The whole apparatus is so costly that the ordinary people cannot afford; (5) The operation interface is not user-friendly; and (6) Owing to the above-mentioned reasons, a more common situation is that most of the insomniacs do not look for solutions to the insomnia through public channel, but search for methods to resolve the issue by themselves instead, which is not only time-consuming but also not necessary ending up with the right solutions. Thus, there is a large gap between the current sleep assistant device and those insomniacs. The ordinary uses are not easy to be acquainted with those sleep assistant device. The sleep assistant devices are hard to be common in people's daily life. Most of the insomniacs do not use those devices.

### SUMMARY OF THE INVENTION

To overcome the abovementioned issues, the present invention aims to provide an electrical sleep assistant device which is compact, economic, easy to handle, being able to assist the insomniac to resolve the issue and easy to be acquainted with. The present invention integrates the biosensors that originally disposed at separate locations and a personal computer equipped with a sleep assistant module into one handheld apparatus, to provide a handheld sleep assistant device. Particularly, the design of a handheld sleep assistant device is appropriate for easy holding and carrying. The handheld sleep assistant device completely eliminates those deficiencies existed in the prior art, and can be fit into people's daily life. The user can carry the sleep assistant device. The sleep coach device is able to collect the user's physiological information at any time, analyze the collected physiological information, perform sleep diagnosis at a medical level, initiate a personal plan for sleep improvement, and assist to implement the plan. Via appropriate interface, the handheld sleep assistant device can control the environmental control equipment, such as air condition, music/stereo systems and lighting, to form a sleeper's central dormitory.

In accordance with one aspect of the present invention, an electrical sleep assistant device, which comprises a handheld case, a display panel, a plurality of biosensors, and a calculation module, is provided. The case has a surface. The display panel is disposed on the surface of the case. The plurality of biosensors are disposed on the surface of the case for collecting a plurality of physiological information. The calculation module is disposed in the case, and coupled to the display panel and the plurality of biosensors. Preferably, a plurality of buttons disposed on the surface of the case, and coupled to the calculation module.

Preferably, a power supply device for providing an electrical power to drive the calculation module, the display panel and the plurality of biosensors.

Preferably, the power supply device is a rechargeable battery and the rechargeable battery is one selected from a group consisting of a lithium-ion battery, a nickel-cadmium battery and a Ni-metal-hydride battery.

Preferably, an earphone socket or a universal serial bus socket is disposed on the surface of the case, and coupled to the calculation module.

Preferably, the calculation module analyzes the plurality of physiological information, outputs an analyzed result to the display panel, and receives the plurality of physiological information from the plurality of biosensors.

Preferably, the calculation module is an electronic device including at least one of a calculation processor and a central processing unit, and a sleep assistant program is installed in the calculation module.

Preferably, the display panel is one selected from a group consisting of a touch-screen panel, a flat-panel display, and a liquid-crystal display.

Preferably, the plurality of biosensors include one selected from a group consisting of a biosensor for heart beat measurement, a biosensor for body temperature measurement, a biosensor for blood pressure measurement, a biosensor for skin conductivity measurement, a biosensor for breath frequency measurement and a combination thereof.

Preferably, the plurality of physiological information include one selected from a group consisting of a heart beat, a body temperature, a blood pressure, a skin conductivity, a breath frequency and a combination thereof.

Preferably, the device has a type being one selected from a group consisting of a handheld apparatus, a personal digital assistance and a pocket-type personal computer.

In accordance with another aspect of the present invention, an electrical sleep assistant device is provided. The device comprises a main body, a display panel, a plurality of biosensors, and a calculation module. The main body has a surface. The display panel is disposed on the surface of the main body. The plurality of biosensors are disposed on the surface for collecting a plurality of physiological information from a user. The calculation module is disposed in the main body, and coupled to the display panel and the plurality of biosensors.

Preferably, a sleep assistant program is installed in the calculation module, the plurality of physiological information include one selected from a group consisting of a heart beat, a body temperature, a blood pressure, a skin conductivity, a breath frequency, and a combination thereof, and the calculation module receives the plurality of physiological information from the plurality of biosensors.

In accordance with a further aspect of the present invention, a method for assisting in sleeping is provided. The method comprises steps of collecting a physiological information of a user via an electrical sleep assistant device and implementing a sleep-coaching program with the electrical sleep assistant device. Preferably, the sleep-coaching program analyses the physiological information to provide a sleep knowledge to the user, and performs an environment modulation for the user based on the physiological information, and the electrical sleep assistant device is a hand-held device.

Preferably, the step of collecting the physiological information of the user further comprises collecting the physiological information with a plurality of biosensors on a surface of the electrical sleep assistant device.

Preferably, the sleep-coaching program comprises a sorting module, a sleep diary management module, a suggestion module, a training module and an environment control module.

In accordance with a further aspect of the present invention, an apparatus for performing the abovementioned method is provided.

Additional objects and advantages of the invention will be set forth in the following descriptions with reference to the accompanying drawings, in which:

### BRIEF DESCRIPTION OF THE DRAWINGS

FIG. 1 is a schematic diagram of the outer appearance of the electrical sleep assistant device according to a preferred embodiment of the present invention;

FIG. 2 is a schematic diagram showing the use of the electrical sleep assistant device according to a preferred embodiment of the present invention;

FIG. 3 is a schematic diagram showing the structure of the sleep coaching program according to a preferred embodiment of the present invention;

FIG. 4 is a schematic diagram showing a user's central dormitory according a preferred embodiment of the present invention;

FIG. 5 is a schematic diagram indicating the collaboration between medical sleep coaching/assistant equipment and the electrical sleep assistant device according to a preferred embodiment of the present invention;

FIG. 6 is a schematic diagram showing the operation interface of the screening module according to a preferred embodiment of the present invention;

FIG. 7 is a schematic diagram showing the operation interface of the training module according to a preferred embodiments of the present invention; and

FIG. 8 is an operation flow chart according to a preferred embodiment of the present invention.

### DETAILED DESCRIPTION OF THE PREFERRED EMBODIMENT

The embodiments described below are merely exemplary and are not intended to limit the invention to the precise forms disclosed. Instead, the embodiments were selected for description to enable one of ordinary skill in the art to practice the invention.

### Embodiment I: Handheld Electrical Sleep Assistant Device

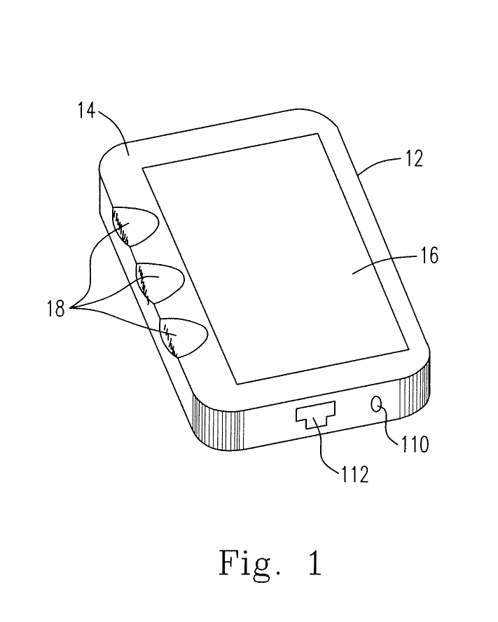

Refer to FIG. 1, which is a schematic diagram of the outer appearance of the electrical sleep assistant device provided by the present invention. The electrical sleep assistant device 10 comprises a case body 12, a surface 14, a display panel 16, biosensors 18, an earphone socket 110, a USB socket 112 and an electronic calculation module (inside the case body 12, therefore not shown in the diagram). The electronic calculation module is a hardware that has functions including logical calculation and digital data memory. In this embodiment, the electronic calculation module is equipped with an electronic circuit having one of a processor and a central processing unit. A sleep coaching program is installed in the electronic circuit. The main usage of the case body 12 is for accommodating the electronic calculation module and relevant components/assemblies, and for protecting the electronic calculation module and relevant components/assemblies. The display panel 16 is disposed on the surface 14 of the case body 12. The plurality of biosensors 18 and/or a plurality of buttons (not shown) are disposed at the lateral sides of the display panel 16. In this embodiment, those biosensors 18 and the display panel 16 are disposed at the surface 14 in parallel. When designing the case body 12, one may use his/her ability of innovation to choose a shape of the outer appearance from one selected from a group consisting rectangular, elliptic, cubic, spherical and columnar, as long as the shape of the case body 12 is applicable for a user to operate the electrical sleep assistant device 10 while holding the device by hand. The case body 12 is made of a material selected from a group consisting of metal alloy, plastic and carbon fibber, which provides sufficient strength to protect the electronic calculation module and the relevant components/assemblies including an electrical power supply. The electrical power supply includes a rechargeable battery, such as a lithium-ion battery, a nickel-cadmium battery and a Ni-metal-hydride battery, to provide electric power to drive the electronic calculation module, the panel display 16 and those biosensors 18. In this embodiment, a 5 volt /12 mA lithium-ion battery is utilized. The panel display 16 is one selected from a group consisting of a touch-screen panel, a flat-panel display, and a liquid-crystal display. In this embodiment, a touch-screen panel display is utilized. The type of the biosensors 18 is one selected from a group consisting of a biosensor for heart beat measurement, a biosensor for body temperature measurement, a biosensor for blood pressure measurement, a biosensor for skin conductivity measurement, a biosensor for breath frequency measurement and a combination thereof.

### Embodiment II: A Method for Assisting in Sleeping

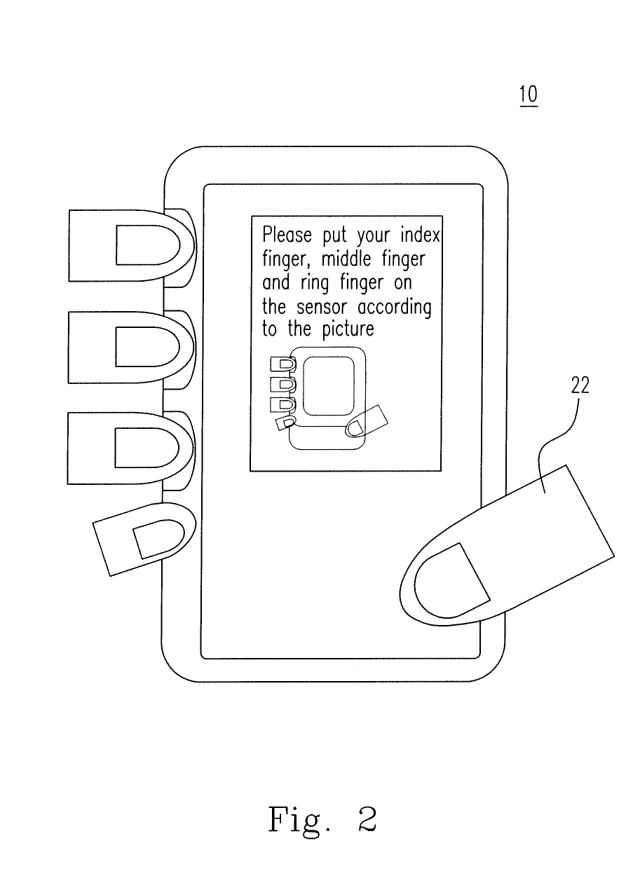

Please refer to FIG. 2, which is a schematic diagram showing the operation of the electrical sleep assistant device 10. In this embodiment, the electrical sleep assistant device is designed for right hand to hold. According to FIG. 2, the user has to hold the electrical sleep assistant device 10 and put each of her/his fingers 22 on each of the biosensors 18 (not shown). When pressed, as FIG. 2 shows, the biosensors begin to collect the user's physiological information including heart beat, body temperature, blood pressure, skin conductivity, breath frequency and a combination thereof, and transmit those physiological information to the sleep coaching program installed in the electronic calculation module. The sleep coaching program is able to interact with the user via the display panel. The sleep assistant device 10 can also be designed for left hand users in other embodiments.

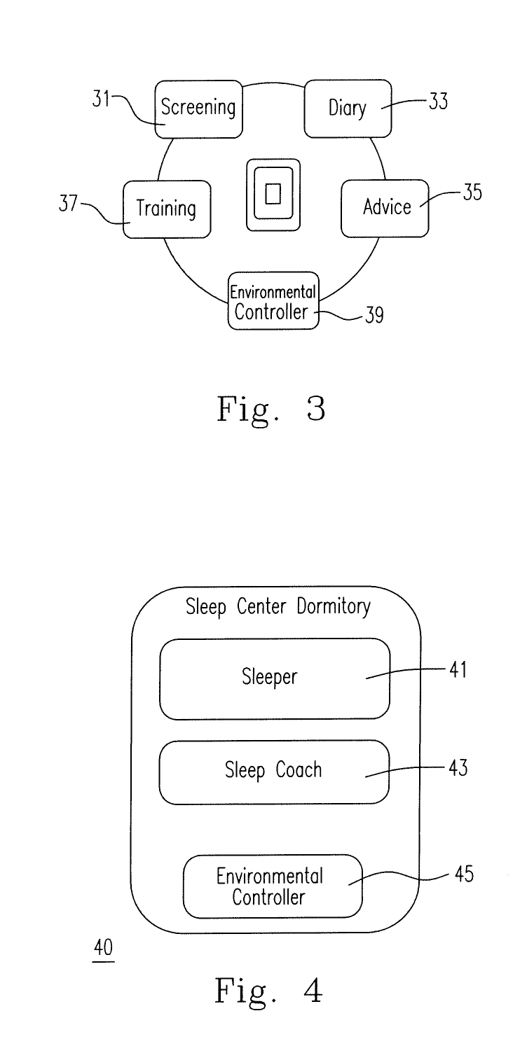

The sleep coaching program installed in the sleep assistant device 10 includes at least 5 sub-programs: a screening module, a sleeping diary management module, an advice module, a training module and an environment controller module. FIG. 3 is a schematic diagram showing the structure of the sleep coaching program. Refer to FIG. 3, the sleep coaching program comprises the screening module 31, the sleeping diary management module 33, the advice module 35, the training module 37 and the environment controller module 39.

The screening module 31 is for collecting a variety of the user's sleep information. Based on the information, the sleep coaching program is cable to verify whether the user is suffering insomnia and the severity of the insomnia if it exists. The screening module 31 takes advantage of electronic questionnaires to investigate the users' fundamental sleep information, including age, sex, health condition, working style, family condition, going abroad condition, life style, sleeping style as well as quality, health behaviour during sleeping, previous strategy of handling insomnia issue, investigation for past sleep history, the quality of life and etc. The sleep coaching program shows a list of questions on the display panel 16 for the user to answer. When the user has completed the answers, the sleep coaching program grades on the answers of the user and shows the grading as well as its meaning on the display panel 16.

For instance, the following is the diagnostic assessment of the screening module 31.

If the user's grading falls in the range from 0 to 7, it means the result does not comply with the definition of insomnia according to clinical diagnostics, in other words, the user is free of insomnia issue;

if the user's grading falls in the range from 8 to 14, it means the result is near the insomnia according to clinical diagnostics;

if the user's grading falls in the range form 15 to 21, it means the result complies with the definition of median insomnia according to clinical diagnostics; and

if the user's grading falls in the range form 22 to 28, it means the result complies with the definition of serious insomnia according to clinical diagnostics.

The screening module 31 identifies possible insomniacs via the abovementioned criteria.

The sleep diary management module 33 is for recording the user's sleep information for a long term. The sleep diary management module 33 requests the user to write a sleep diary. The contents of the sleep diary include two portions, the information before sleeping and the information after waking up.

As for the information before sleep, the following list of questions are to be answered by the user:

1. What is the bedtime?

2. Have you ever taken a nap in the daytime? When?

3. Did you drink alcoholic beverage today?

4. Did you drink beverage containing caffeine today?

5. Your degree of awaken at noon

6. Your degree of awaken in the afternoon

As for the information after waking up, the following list of questions are to be answered by the user:

1. What is the wakeup time?

2. What is the time period of getting into sleep?

3. How many times did you wake up?

4. What is the feeling after waking up?

The sleep coach program utilizes the mentioned sleep diary to trace the user 's sleeping condition, and provides advices regarding sleep or more appropriate sleep coaching training, based on the information.

If the user is considered an insomniac after the screening, the advice in the sleep coaching program takes advantage of cognitive behaviour therapy to the user as appropriate. The cognitive behaviour therapy comprises sleep restriction therapy, stimulus control instruction, cognitive therapy, sleep hygiene and relaxation training. The selection of methodology is based on the grading of the screening module. Appropriate therapies are adopted for different degree of the insomnia issues.

For instance, if the sleep restriction therapy is adopted, the sleep coaching program requests the user follow the described life style as below:

1. the bed time should be one o'clock in the morning; and the wakeup time is seven o'clock in the morning;

2. sleeping on the bed during the other time period is not allowed;

3. to continue the writing of sleep diary; and

4. in the beginning, one might feel a bit of lack of sleep, however, implementing the mentioned method will be helpful to increase the stability of sleep.

The advice module 35 will combine assessments including physiological information collected from the user and the sleep diary written by the user to diagnose the user's sleep condition for additionally providing treatment suggestions and professional medical information, which includes providing the information regarding professional medical organizations and a variety of therapies. The advice module 35 also provides the user the information of professional medical organizations and further more make reservations for clinic for the user.

The training module 37 comprises relaxation training. The sleep coaching program guides the user to inhale and exhale. In this embodiment, when the user tends to adopt the relaxation training, he/she may connect the electrical sleep assistant device 10 to a personal computer. When the user inhales and exhales with his/her mouth toward the biosensors, the biosensors measures the time of the inhaling and exhaling, and provides suggestions to the user for improvement to complete the relaxation training.

For instance, in the procession of relaxation training, the following operation instruction may be displayed: “Please pay attention to your breath, inhale slowly, and exhale slowly.”

Or the following instruction for exercising may be displayed: “The longer the inhaling time, the more flowers are shown on the screen. When exhaling, the longer the exhaling time, the farther the flowers are blown away. Try it!!!”

The mentioned inhaling time is collected by the biosensors. During the process of relaxation training, the electrical sleep assistant device will provide notifications such as “Please pay attention to your breath, inhale slowly, and exhale slowly.” the user when appropriate.

The environmental controlling module 39 refers to the physiological information collected from the user, the sleep diary written by the user and the user's preference of sleep, and adjusts several environment conditions that might effect to the sleep quality, in which the environment conditions include sound, light, temperature and air quality of the dormitory. Additionally, lighting and sound control for the architecture environment is adopted by the environmental controlling module 39, to construct an environment which is favorable for sleep and to achieve an environmental condition dedicated to the sleeper for building the optimized sleep environment. The environmental controlling module 39 effectively assists the treatments for sleep disorder, and constructs a user's central dormitory. Refer to FIG. 4, which is a schematic diagram showing a user's central dormitory 40 constructed according to the present invention. According to FIG. 4, the central dormitory 40 comprises a sleeper 41, an electronic sleep coach 43 and an environmental controller module 45.

### Embodiment III: Collaborating with External Apparatus

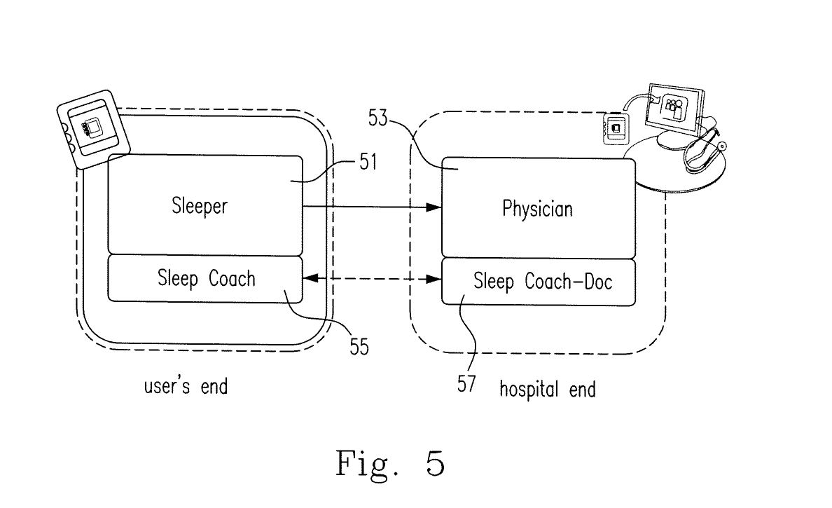

The electrical sleep assistant device provided by the present invention is able to further be collaborated with medical sleep coaching/assistant equipment. Refer to FIG. 5, which is a schematic diagram indicating the collaboration between medical sleep coaching/assistant equipment and the electrical sleep assistant device provided by the present invention. FIG. 5 shows a user (sleeper) 51, a physician 53, an electrical sleep assistant device (sleep coach) 55 and a medical sleep coaching/assistant equipment (sleep coach-doc) 57, in which the medical sleep coaching/assistant equipment 57 is disposed in a personal computer at the hospital end.

The electrical sleep assistant device 55 provided by the present invention is for the sleeper 51 to use at home, while the medical sleep coaching/assistant equipment 57 is for the physician 53 to use at medical organizations. The operation interface of the medical sleep coaching/assistant equipment 57 is particularly for the use in hospitals, which is different with that of the electrical sleep assistant device 55. The sleeper 51 may carry the electrical sleep assistant device 55 to a professional medical organization to look for clinical treatment from the physician 53, periodically or not. When doing the clinic, the electrical sleep assistant device 55 of the user's end can be connected to the medical sleep coaching/assistant equipment 57 (for example, via the USB socket 112 of the electrical sleep assistant device 10). The electrical sleep assistant device 55 provides those user's sleep information long-term collected at the user's end, such as the daily sleep diary, the sleep training process and effect, plurality of physiological information collected by the biosensors and the answers to the questionnaire, to the medical sleep coaching/assistant equipment 57. During the clinic, the physician 53 can perform a more accurate diagnose, based on those comprehensive long-term record.

For instance, the user's sleep information collected at the user end by the electrical sleep assistant device in long-term efforts includes the following information:

Individual information
1. Sleep style:
1.1 Overall sleeping time span in average: 5 hours
1.2 Average time span for getting asleep: 2.1 hours
1.3 Average sleep efficiency: 58%
1.4 Waking up time in average: 7 o'clock
2. Physiological condition: no particular physiological issue
3. Psychological condition:
3.1 Beck Depression Inventory rated 5, not compliance
3.2 . . .
4. Environment condition:
4.1 Children
4.2 Pats

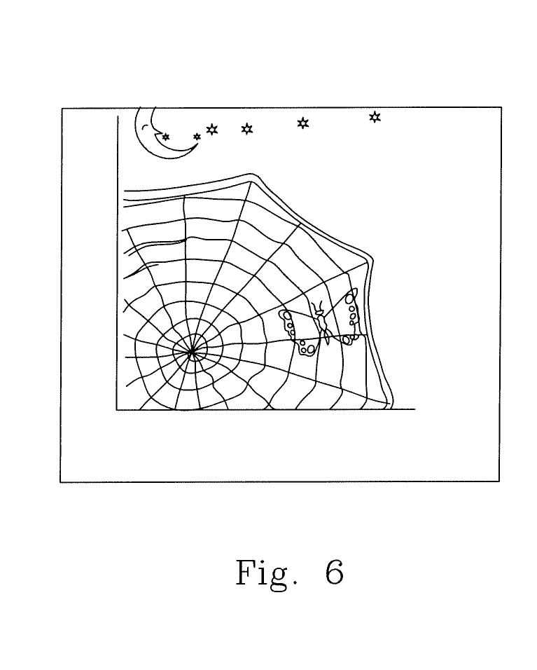

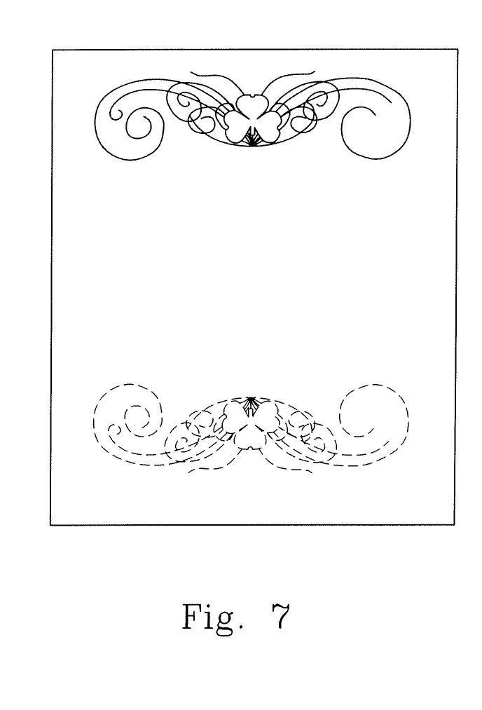

Refer to FIG. 6, which is a schematic diagram showing the operation interface of the screening module of the electrical sleep assistant device. Refer to FIG. 7, which is a schematic diagram showing the operation interface of the training module of the electrical sleep assistant device.

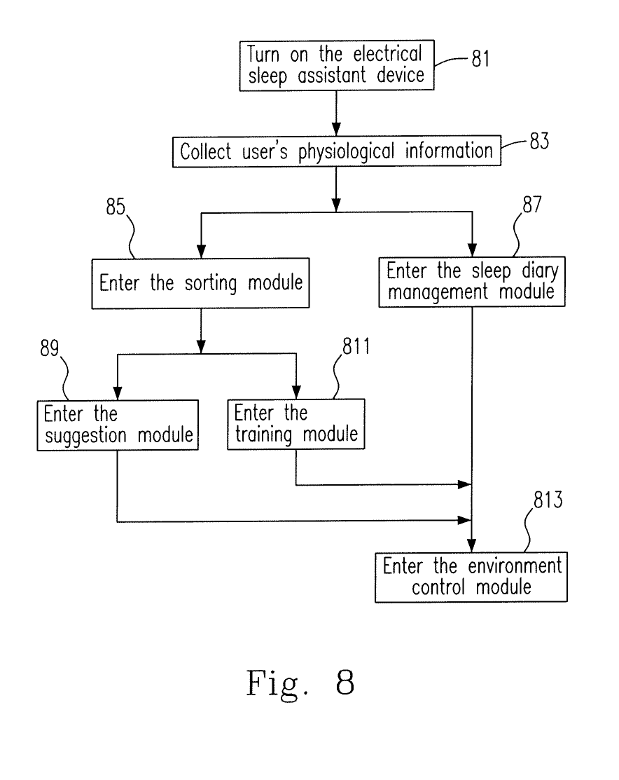

Refer to FIG. 8, which is an operation flow chart according to a preferred embodiment of the present invention. In accordance with FIG. 8, the operation flow includes Step 81 turning on the electrical sleep assistant, Step 83 collecting the user's collecting physiological information, Step 85 entering the screening module, Step 87 entering the sleep diary management module, Step 89 entering the advice module, Step 811 entering the training module and Step 813 entering the environmental controller module.

When a user who potentially suffers insomnia, no matter long term or shot term, he/she should in general follows the procedure set forth hereinafter to operate the electrical sleep assistant device provided by the present invention. Firstly, the user turn on the electrical sleeping assistant device and enter a main screen, which is the Step 81. Then, the user puts her/his fingers on the biosensors, and the electrical sleeping assistant device starts to collect the user's physiological information, which is Step 83. If the user is a first-time user, she/he may choose to enter the screening module to diagnose the level of insomnia, which is the Step 85. If the user is not a first-time user, she/he may directly choose to enter the sleep diary management module to write the sleep diary, which is the Step 87. Upon completion of implementing the screening module, the user may choose either to enter the advice module firstly, to obtain the advices provided by the electrical sleeping assistant device to the user based on the various sleep information of the user, which is the Step 89, or to enter the training module and follow the guidance of the electrical sleeping assistant device to implement the different kinds of sleep assistant training, which is the Step 811. Finally, the user may choose to the environment controller module, in which the electrical sleeping assistant device adjust and control those environmental factors that might affect to the sleeping quality, based on the physiological information collected from the user, the sleep diary written by the user, the user's preferred sleep condition and etc. Actually, the implementation of those steps or modules is not necessary exact the same with the mentioned procedure. The mentioned procedure merely provides a suggestion for the operation flow. The user also may choose to directly enter anyone of the modules. The operation flow of FIG. 8 is only a feasible one. The user is not restricted to follow the operation flow provided by FIG. 8 only.

Based on the above, the present invention provides an electrical sleep assistant device which is compact, economic, easy to handle, being able to assist the insomniac to resolve the issue and easy to be acquainted with. Thus, it is apparent that the present invention has advantages over the prior art.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims that are to be accorded with the broadest interpretation so as to encompass all such modifications and similar structures.

What is claimed is:

1. An electrical sleep assistant device, comprising:
a handheld case having a surface;
a display panel disposed on the surface of the case;
a plurality of biosensors disposed on the surface of the case for collecting a plurality of physiological information; and
a calculation module disposed in the case, and coupled to the display panel and the plurality of biosensors.

2. An electrical sleep assistant device as claimed in claim 1, further comprising a plurality of buttons disposed on the surface of the case, and coupled to the calculation module.

3. An electrical sleep assistant device as claimed in claim 1, further comprising a power supply device for providing an electrical power to drive the calculation module, the display panel and the plurality of biosensors.

4. An electrical sleep assistant device as claimed in claim 3, wherein the power supply device is a rechargeable battery.

5. An electrical sleep assistant device as claimed in claim 4, wherein the rechargeable battery is one selected from a group consisting of a lithium-ion battery, a nickel-cadmium battery and a Ni-metal-hydride battery.

6. An electrical sleep assistant device as claimed in claim 1, further comprising an earphone socket disposed on the surface of the case, and coupled to the calculation module.

7. An electrical sleep assistant device as claimed in claim 1, further comprising a universal serial bus socket disposed on the surface of the case, and coupled to the calculation module.

8. An electrical sleep assistant device as claimed in claim 1, wherein the calculation module analyzes the plurality of physiological information, outputs an analyzed result to the display panel, and receives the plurality of physiological information from the plurality of biosensors.

9. An electrical sleep assistant device as claimed in claim 1, wherein the calculation module is an electronic device including at least one of a calculation processor and a central processing unit, and a sleep assistant program is installed in the calculation module.

10. An electrical sleep assistant device as claimed in claim 1, wherein the display panel is one selected from a group consisting of a touch-screen panel, a flat-panel display, and a liquid-crystal display.

11. An electrical sleep assistant device as claimed in claim 1, wherein the plurality of biosensors include one selected from a group consisting of a biosensor for heart beat measurement, a biosensor for body temperature measurement, a biosensor for blood pressure measurement, a biosensor for skin conductivity measurement, a biosensor for breath frequency measurement and a combination thereof.

12. An electrical sleep assistant device as claimed in claim 1, wherein the plurality of physiological information include one selected from a group consisting of a heart beat, a body temperature, a blood pressure, a skin conductivity, a breath frequency and a combination thereof.

13. An electrical sleep assistant device as claimed in claim 1, wherein the device has a type being one selected from a group consisting of a handheld apparatus, a personal digital assistance and a pocket-type personal computer.

14. An electrical sleep assistant device, comprising:
a main body having a surface;
a display panel disposed on the surface of the main body;
a plurality of biosensors disposed on the surface for collecting a plurality of physiological information from a user; and
a calculation module disposed in the main body, and coupled to the display panel and the plurality of biosensors.

15. An electrical sleep assistant device as claimed in claim 14, wherein a sleep assistant program is installed in the calculation module, the plurality of physiological information include one selected from a group consisting of a heart beat, a body temperature, a blood pressure, a skin conductivity, a breath frequency, and a combination thereof, and the calculation module receives the plurality of physiological information from the plurality of biosensors.

16. A method for assisting in sleeping, comprising steps of:
collecting a physiological information of a user via an electrical sleep assistant device; and
implementing a sleep coaching program with the electrical sleep assistant device.

17. A method for assisting in sleeping as claimed in claim 16, wherein the sleep coaching program analyses the physiological information to provide a sleep knowledge to the user, and performs an environment modulation for the user based on the physiological information, and the electrical sleep assistant device is a hand-held device.

18. A method for assisting in sleeping as claimed in claim 16, wherein the step of collecting the physiological information of the user further comprising:
collecting the physiological information with a plurality of biosensors on a surface of the electrical sleep assistant device.

19. A method for assisting in sleeping as claimed in claim 16, wherein the sleep coaching program comprises a sorting module, a sleep diary management module, a suggestion module, a training module and an environment control module.

20. An apparatus for performing the method as claimed in claim 16.
